(19) United States
(12) Patent Application Publication
(10) Pub. No.: US 2003/0027780 A1
(43) Pub. Date: Feb. 6, 2003

(54) Multiparticulate formulation

(76) Inventors: Gregory E. Hardee, Rancho Santa Fe, CA (US); Lloyd G. Tillman, Carlsbad, CA (US); Maria Gonzalez-Ferreiro, Berlin (DE); Rahul C. Mehta, San Marcos, CA (US); Ching-Leou Teng, San Diego, CA (US)

Correspondence Address: Woodcock Washburn LLP, 46th Floor, One Liberty Place, Philadelphia, PA, 19103, US

(21) Appl. No.: 10/071,822

(22) Filed: Feb. 8, 2002

Related U.S. Application Data
(63) Continuation of application No. 09/256,515, filed on Feb. 23, 1999, now abandoned.

(51) Int. Cl. A61K 48/00; A61K 9/14

(52) U.S. Cl. 514/44; 424/46

(57) ABSTRACT

The present invention is related to non-parenteral multiparticulate formulations capable of transporting therapeutic, prophylactic and diagnostic agents across mucosal membranes such as gastrointestinal, buccal, nasal, rectal and vaginal. Formulations comprise a plurality of carrier particles, an agent to be delivered across a mucosal membrane, and a penetration enhancer. The drug is adhered to the surface of the carrier particle or is impregnated within by electrostatic, covalent or mechanical forces.

### CROSS-REFERENCE TO RELATED APPLICATIONS

This patent application is a continuation of application Ser. No. 09/256,515, filed Feb. 23, 1999, the entirety of which is incorporated herein by reference.

### FIELD OF THE INVENTION

The present invention is related to pharmaceutical formulations, in particular non-parenteral pharmaceutical formulations capable of delivering therapeutic and diagnostic agents across mucosal membranes.

### BACKGROUND OF THE INVENTION

Parenteral administration of pharmaceutical agents or drugs by injection (intravenous, subcutaneous, intramuscular) while common and necessary under certain circumstances is not the most desirable route from a patient standpoint. Because it can seldom be performed by the individual in need thereof and requires assistance of professional care givers it is an inconvenient and costly route. Further, there is often associated discomfort at the site of administration and there is always an inherent risk of infection. Not surprisingly, patient compliance is much greater for drugs administered non-parenterally, in particular, oral, nasal and pulmonary administration. For their convenience and non-invasive nature, these non-parenteral routes of administering drugs are preferred by patients.

However, each of these involves transport of the drug across a mucosal surface or membrane, which comprises an epithelium and a mucus secretion thereon. The mucus secretion of a mucosal membrane presents a barrier to protect the membrane from physical damage as well as to prevent undesired substances from passing through and entering epithelial tissue or the lymph system or the blood stream. Unfortunately, mucus membranes can also prevent significant uptake of some drugs such as those having large molecular weight, or are proteinaceous or are nucleic acids. Consequently these drugs are most often administered to individuals parenterally, for example, injected intravenously, subcutaneously or intramuscularly.

It would therefore be desirable to provide a convenient formulation for transporting pharmaceutical agents across mucosal membrane.

### SUMMARY OF THE INVENTION

In accordance with an aspect of the present invention there is provided a multi-particulate formulation or composition comprising a plurality of carrier particles; a biologically active substance to be delivered across a mucosal membrane, wherein said biologically active substance is bound to said carrier particles; and a penetration enhancer.

In another aspect of the invention there is provided a method of delivering a biologically active substance across a mucosal membrane, by introducing to the mucosal membrane a multi-particulate formulation comprising a plurality of carrier particles; the biologically active substance and a penetration enhancer, wherein said biologically active substance is bound to said carrier particles.

### BRIEF DESCRIPTION OF THE DRAWINGS

FIG. 1 is a graph showing concentration of antisense oligonucleotide in plasma at time intervals following administration of a multiparticulate formulation.

### DETAILED DESCRIPTION OF THE INVENTION

A multi-particulate formulation or composition is provided having a plurality of carrier particles; a biologically active substance (BAS) to be delivered across a mucosal membrane, and a penetration enhancer, wherein said biologically active substance is bound to said carrier particles. Formulations of the invention associate with mucosal membranes such as buccal, nasal, pulmonary, gastrointestinal and vaginal, thereby transporting biologically active substances to an individuals lymph system, blood stream or epithelial tissue.

Carrier Particles

Carrier particles according to the present invention include a variety of particle-forming substances that are preferably capable of maintaining a biologically active substance (BAS) in intimate association with mucosal membranes thereby enhancing transport of the BAS across mucosal membranes. Preferred carrier particles are those which enhance bioavailability of biologically active substances upon administration and delivery to a mucosal membrane. “Bioavailability” in this context is the percentage of the total amount of HAS administered that is found in plasma, epithelial tissue or target tissue at a given time post administration. For enhancing bioavailability of a BAS, it is preferred that carrier particles are composed of material that resists degradation prior to contacting a mucosal membrane. Carrier particles, depending on their chemical composition and mode of preparation, include a variety of regular or irregular shapes and sizes and may be a solid or a gel. For example, preferred carrierparticles are generally spherical (hollow or filled) having millimeter (greater than about 1 mm), micron (greater than about 1μ) or nanometer (greater than about 10 nm) diameter and are thus referred to as miniparticles (tablets), microparticles and nanoparticles respectively. Preferred carrier particles have a diameter of about 0.01 to 1000μ. More preferably carrier particles are about 0.1 to 500μ, and more preferably 1 to 300μ.

Preferred particle-forming substances include poly-amino acids; polyimines; polyacrylates; dendrimers; polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches;

By the term “bound” is meant that biologically active substances are associated with carrier particles by way of electrostatic (ionic, polar, Van der Waals), covalent or mechanical (non-electrostatic, non-covalent) interaction depending on the composition of the BAS and carrier particle as well as the method of preparing the carrier particle.

For example, an anionic BAS such as an oligonucleotide can be bound to cationic carrier particles by ionic interaction. In a particularly preferred embodiment, particle-forming substances are polycationic polymers such as chitosan, poly-L-lysine, polyhistidine, polyornithine, polyspermines, protamine, polyvinylpyridine, polythiodiethylamino-methylethylene P(TDAE), polyaminostyrene (e.g. para-amino), poly(methylcyanoacrylate), poly(ethylcyanoacrylate), poly(butylcyanoacrylate, poly(isobutylcyanoacrylate), poly(isohexylcynaoacrylate), DEAE-methacrylate, DEAE-hexylacrylate, DEAE-acrylamide, DEAE-albumin and DEAE-dextran. In a particularly preferred embodiment, the particle-forming substance is chitosan. In another particularly preferred embodiment, the particle-forming substance is poly-L-lysine complexed with alginate. In a further embodiment, formulations of the invention comprise cationic carrier particles and anionic biologically active substances, such as oligonucleotides, associated thereto by ionic interaction.

In an alternative embodiment, particle-forming substances are non-polycationic i.e. carry an overall neutral or negative charge, such as polyacrylates, for example polyalkylacrylates (e.g. methyl, hexyl etc.), polyoxethanes, poly(DL-lactic-co-glycolic acid (PLGA), and polyethyleneglycol (PEG). In a particularly preferred embodiment, the particle forming substance is PLGA.

In another embodiment, carrier particles further comprise an outer coating. Said outer coating may be a material capable of associating a BAS to the carrier particle, for example, a cationic polymer which binds an anionic BAS, or be a protective material resisting degradation in biological environments such as in the stomach, lumen, plasma or cytoplasm. In a particular embodiment, carrier particles may be substantially coated on their outer surface with lipid compounds as described in WO98/29,557, WO98/29,099, WO98/04,719, WO97/12,618 and WO92/21,330 incorporated herein by reference. The lipid coating serves to enhance cellular membrane fusion and therefor cellular uptake of particles.

Further, carrier particles may have attached thereto targeting molecules which serve to bind the particle to the mucosal membrane cells and/or to direct the particle once across the mucosal membrane to a particular cell, tissue or organ type of interest. Targeting molecules may be peptidic such as proteins or peptides or small molecules. Protein targeting molecules include antibodies which selectively bind to antigenic determinants that are predominant at the site of interest. Protein and peptide targeting molecules are preferably those that are selective ligands for cell surface receptors. For example, certain growth factors such as EGF (epidermal growth factor) and PDGF (platelet derived growth factor) are overexpresssed on the surface of certain cancer cells. The proteins EGF and PDGF therefore serve as a suitable targeting molecule for directing carrier particles containing anticancer agents. More preferably, targeting molecules are peptide fragments of proteins which bind to cellular receptors. Similarly, certain small organic molecules are ligands for cell surface receptors. For example, folic acid receptors are known to be overexpressed in certain cancer cells. Consequently folate is a useful targeting molecule for delivering anticancer agents to cancer cells. Targeting molecules may be linked to carrier particles of the invention by a linking group attached to a functional group of the carrier particle. Suitable linking groups include peptides, hydrocarbon chains such as alkyl, or other polymers. A particularly preferred linking group is polyethylene glycol (PEG) of approximately 1 to 250 repeating units, preferably about 20 to 100 repeating units and more preferably 70 to 80 repeating units.

Biologically Active Substances

In accordance with present invention “biologically active substances” (BAS) include a wide variety of substances having pharmacological effect (therapeutic, prophylactic or diagnostic) in animals such as mammals, in particular humans. Types of biologically active substances which may be employed include small organic molecules, macromolecules and polymers such as peptides, proteins, monoclonal antibodies and fragments thereof, nucleic acids such as nucleosides, nucleotides, single stranded oligonucleotides (probes, antisense, ribozymes), double stranded oligonucleotides (vectors, plasmids). The present invention is particularly useful for transporting proteinaceous and oligo(ribo/deoxy)nucleic acids across mucosa. In a particular embodiment, oligonucleotides are employed in formulations of the invention, in particular single stranded oligonucleotides such as those having antisense or ribozyme activity. Oligonucleotides include those incorporating naturally-occurring structure i.e. 3′-5′ phosphodiester linked ribo or deoxyribonucleosides or those incorporating non-naturally occurring features. For example, one or more backbone linkages of oligonucleotides may be other than naturally occurring phosphodiester, for example, phosphotriester, phosphorothioate, phosphorodithioate, phosphonates (H, alkyl, aryl etc.), boranophosphate, selenophosphate, ethylene glycol, methylenemethylimino (MMI) and others. Other backbone modifications include 2′-5′ backbone linkages and those having an acyclic sugar-backbone structure such as Peptide Nucleic Acids (PNA's) wherein the sugar and phosphate components are replaced with a peptidic structure.

The sugar component of oligonucleotides may be modified to include hexoses, cyclopentyl or cyclohexyl as well as various substituents at the 2′ position including halogen, alkoxy (2′-O-alkyl), alkoxyalkoxy (2′-O-alkyl-alkoxy) and derivatives thereof.

Particularly preferred 2′ substituents include methoxy, methoxyethoxy (MOE), aminooxyethoxy (AOE) and dimethylaminooxyethoxy (DMAOE). Other non-natural oligonucleotide modifications include base modifications such as 5-methyl-cytosine and 2-aminoadenine and base or sugar functionalization such as cholesterol, intercalators and targeting molecules such as receptor ligands, peptides, antibodies and folic acid. Examples of specific oligonucleotides which may be employed in formulations of the present invention include:

| | | |
|---|---|---|
| ISIS-5132 | TCCCG CCTGT GACAT GCATT | (SEQ ID NO:1) |
| ISIS-2302 | GCCCA AGCTG GCATC CGTCA | (SEQ ID NO:2) |
| ISIS-2922 | GCGTT TGCTC TTCTT CTTGC G | (SEQ ID NO:3) |
| ISIS-3521 | GTTCT CGCTG GTGAG TTTCA | (SEQ ID NO:4) |
| ISIS-2503 | TCCGT CATCG CTCCT CAGGG | (SEQ ID NO:5) |
| ISIS-13312 | GCGTT TGCTC TTCTT CTTGC G | (SEQ ID NO:6) |
| ISIS-5320 | TTGGG GTT | (SEQ ID NO:7) |
| ISIS-14803 | GTGCT CATGG TGCAC GGTCT | (SEQ ID NO:8) |
| ISIS-28089 | GTGTG CCAGA CACCC TATCT | (SEQ ID NO:9) |

wherein (i) each oligo backbone linkage is a phosphorothioate linkage and (ii) nucleosides in bold font incorporate a 2′ 0 methoxyethyl modified sugar and iii) underlined cytosine nucleosides incorporate a 5-methyl substituent on their nucleobase.

Penetration Enhancers

The present invention employs various penetration enhancers in order to effect the gastrointestinal delivery of biologically active substances (BAS). Penetration enhancers may be classified as belonging to one of five broad categories i) surfactants, ii) fatty acids, iii) bile salts, iv) chelating agents, and (v) non-chelating non-surfactants as described in Lee et al (Critical Reviews in Therapeutic Drug Carrier Systems, 1991, p. 92).

i) Surfactants: In connection with the present invention, surfactants (also known as “surface-active agents”) are chemical entities which, when dissolved in an aqueous solution, reduce the surface tension of the solution or the interfacial tension between the aqueous solution and another liquid, with the result that absorption of biologically active substances through mucosa is enhanced. Surfactants include polyoxyethylene-9-lauryl ether, polyoxyethylene-20-cetyl ether (Lee et al., Critical Reviews in Therapeutic Drug Carrier Systems, 1991, page 92) and perfluorohemical emulsions, such as FC-43 (Takahashi et al., J. Pharm. Pharmacol., 1988, 40:252). Bile salts and acids as well as fatty acids and salts thereof may also be considered to be surfactants,

ii) Fatty Acids: Various fatty acids, derivatives and salts thereof act as penetration enhancers. Suitable fatty acids include, for example, oleic acid, lauric acid, capric acid (a.k.a. n-decanoic acid), myristic acid, palmitic acid) stearic acid, linoleic acid, linolenic acid, dicaprate, tricaprate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines and mono- and di-glycerides thereof and/or physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 1990, 7:1; El-Hariri et al., J. Pharm. Pharmacol. 1992, 44:651). In a preferred embodiment of the present invention fatty acid/salt penentrations enhancers are sodium caprate and sodium laurate.

iii) Bile Acid And Salts: The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile acid” or “bile salt” includes any of the naturally occurring components of bile as well as any of their synthetic derivatives. The bile acids and salts of the present invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA, sodium ursodeoxycholate), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7:1; Yamamoto et al., J. Pharm. Exp. Ther., 1992, 263:25; Yamashita et al., J. Pharm. Sci., 1990, 79:579). In a preferred embodiment, bile penetration enhancers are ursodeoxycholic acid (UDCA) and chenodeoxycholic acid (CDCA). In a more preferred embodiment, bile penetration enhancers are the sodium salts of UDCA and CDCA. Most preferably, the penetration enhancer of the invention is the sodium salt of UDCA.

iv) Chelating Agents: Chelating agents, as used in connection with the present invention, can be defined to be compounds that remove metallic ions from solution by forming complexes therewith, with the result that absorption of oligonucleotide through mucosa is enhanced. In a context wherein the biologically active substance of the invention is an oligonucleotide e.g. an antisense oligonucleotide, chelating agents also serve as inhibitors of nucleases. Most characterized DNA nucleases require a divalent metal ion for catalysis and are thus inhibited by chelating agents (Jarrett, J. Chromatogr., 1993, 618, 315). Chelating agents of the invention include but are not limited to disodium ethylenediaminetetraacetate(EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7:1; Buur et al., J. Control Rel., 1990, 14:43).

v) Non-Chelating Non-Surfactants: As used herein, non-chelating non-surfactant penetration enhancing compounds can be defined as compounds that demonstrate insignificant activity as chelating agents or as surfactants but that nonetheless enhance absorption of oligonucleotide through mucosa (Muranishi, Critical Reviews in Therapeutic Drug Carrier Systems, 1990, 7:1). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39:621). Another compound having penetration enhancing qualities is Zonula occludens toxin (Zot) isolated from Vibrio cholerae. This protein has been shown to regulate intestinal tight junction permeability by a receptor mediated pathway (Fasano et al, Proc Natl Acad Sci USA, 1991, 88 (22):5242; and Gastroenterology, 1997, 112:839). Zot protein or fragments thereof capable of binding and activating this receptor may be co-administered with a BAS alone or in conjunction with carrier particle-bound biologically active substances, particularly when the BAS is an oligonucleotide.

Penetration enhancers are employed in formulations of the invention as an additional component or are incorporated within, the carrier particles. In the former context, penetration enhancers are in any suitable form e.g. powder, gel, solution etc. in which carrier particles are mixed. In the latter context, carrier particles are impregnated or have an outer coating of penetration enhancers covering a substantial portion of the carrier particle surface. Alternatively, penetration enhancers are formed into particles themselves which may be mixed with carrier particles. Regardless of dosage forms employed, it is preferred that penetration enhancers are presented to mucosal membranes prior to or concomitantly with the carrier particles. It is contemplated that penetration enhancers are released from the formulation prior to release of the BAS particle complex or alternatively the penetration enhancer is administered prior to the BAS particle complex.

In a particular embodiment, penetration enhancers useful in the present invention are mixtures of penetration enhancing compounds. For example, a particularly preferred penetration enhancer is a mixture of UDCA (and/or CDCA) with capric and lauric acids or salts thereof e.g. sodium. Such mixtures whether in the context of carrier particle systems of the present invention or otherwise are useful for enhancing the delivery of biologically active substances across mucosal membranes, in particular intestinal mucosa. Preferred penetration enhancer mixtures comprise about 5-95% of bile acid or salt(s) UDCA and/or CDCA with 5-95% combined capric and lauric acid. Particularly preferred are mixtures of the sodium salts of UDCA, capric acid and lauric acid in a ratio of about 1:2:2 respectively.

Penetration enhancers and mixtures thereof are present in formulations of the invention in an amount of about 1-99% relative to the biologically active substance. Actual relative amounts will depend on the particular biologically active substance. For instance, when the biologically active substance is an oligonucleotide e.g. an antisense oligonucleotide, the amount of penetration enhancer or mixture employed is from about 40 to 95%, preferably 50 to 90%.

Bioadhesive

In an embodiment of the invention formulations further comprise a bioadhesive material which serves to adhere carrier particles to a mucosal membrane. Preferably, carrier particles are themselves bioadhesive, as is the case with PLL-alginate carrier particles, or may be coated with a bioadhesive material. Such materials are well known in the formulation art, examples of which are described in WO 85/02,092 incorporated herein by reference. Preferred bioadhesive materials include polyacrylic polymers (e.g. carbomer and derivatives of carbomer), tragacanth, polyethyleneoxide cellulose derivatives (e.g. methylcellulose, carboxymethylcellulose, hydroxypropylmethylcellulose (HPMC), hydroxyethylcellulose (HEC), hydroxypropylcellulose (HPC) and sodium carboxymethylcellulose (NaCPC)), karya gum, starch, gelatin and pectin.

Mucolytic

In another embodiment of the invention, formulations further comprise a mucolytic substance which serves to degrade or erode mucin, in part or completely, at the site of the mucosal membrane to be traversed. Mucolytic substances are well known in the formulation art and include N-acetylcysteine, dithiothreitol, pepsin, pilocarpine, guaifenesin, glyceryl guaiacolate, terpin hydrate, ammonium chloride, guattenesin, ambroxol, bromhexine, carbocysteine, domiodol, letosteine, mecysteine, mesna, sobrerol, stepronin, tiopronin and tyloxapol.

In another aspect of the invention there is provided a method of delivering a biologically active substance across a mucosal membrane, comprising introducing to the mucosal membrane a multi-particulate formulation according to the invention.

It is generally desirable to have as high a weight ratio of BAS to carrier particle as possible consistent with particle stability. The amount of pharmaceutical agent will vary depending on the nature and composition of the agent but in general will be from about 1:10 to about 1:1000.

According to an aspect of the invention, a BAS is delivered across a mucosal membrane in an animal, in particular humans, by administering a formulation of the invention to the animal. Administration is non-parenteral e.g. oral, rectal, enema, vaginal, buccal, sublingual, nasal or by pulmonary inhalation. The dosage form used will depend on route of administration, the type of therapeutic, prophylactic or diagnostic indication. The dosage forms include solutions, suspensions, emulsions, ointments, gels, tablets, capsules, gelcaps, sachets, troches, sprays, beads (immediate or time release) and SECs (soft elastic capsules or “caplets”). In a preferred embodiment, formulations of the invention are administered orally.

Other components of formulations include dyes, thickeners, plasticizers, flavoring agents, diluents, emulsifiers, disintegrants and binders. Disintegrants and binders include EMDEX, PRECIROL and AVICEL. The formulation can also include materials effective in protecting the biologically active substance from pH extremes of the stomach, or in releasing the nucleic acid over time, to optimize the delivery thereof to the gastrointestinal mucosa. Enteric coatings for acid-resistant tablets, capsules and caplets are known in the art and include cellulose acetate phthalate (CAP), propylene glycol, EUDRAGIT and sorbitan monoleate. Various methods for producing formulations with these components are well known in the art (see Remington's Pharmaceutical Sciences, 18th Ed., Gennaro, ed., Mack Publishing Co., Easton, Pa., 1990).

The amount of biologically active substance administered will depend on the route of administration, the indication being treated as well as the individual being treated. For antisense oligonucleotides, the amount will range from about 0.01 mg to 100 g per kg body weight, several times per day to yearly.

Poly-L-lysine/alginate multiparticulate formulations are prepared according to techniques known by the skilled artisan. A particular general method is as follows. Sodium alginate is mixed with calcium chloride in water to form a calcium alginate pregel. Poly-L-lysine and a biologically active substance (BAS) is mixed in water and then added to the pregel and mixed to form a multiparticulate suspension. Alternatively, poly-L-lysine is added to the pregel thereby forming a multiparticulate suspension and subsequently adding BAS to the suspension.

PLGA multiparticulate formulations are prepared according to techniques well known to those skilled in the art. A particular general method is as follows. PLGA polymer and oil soluble components are dissolved in an organic solvent and water soluble components are dissolved in water. The biologically active substance (BAS) to be administered is dissolved in either the polymer solution or the aqueous solution as appropriate. The two solutions are combined and mixed thoroughly to give a dispersed phase. A continuous phase is prepared by dissolving a surfactant in a solvent such as water, mineral oil, heptane, octane, and cottonseed oil. A dispersion is then prepared by slowly adding the dispersed phase to the continuous phase while mixing. Temperature is then increased and volatile solvents are allowed to evaporate. The resulting multi-particulates may then be recovered by filtration from the solution.

### EXAMPLE 1

poly-L-lysine/alginate Particles with Oligonucleotide ISIS-3521 (SEQ ID NO: 4)

Formulation 1

Two solutions, one of sodium alginate (240 mg), medium viscosity dissolved in approximately 350 mL of distilled H₂O and the other 52.96 mg of CaCl2.2H₂O in approximately 50 mL of dH₂O were combined to give a calcium alginate pregel (0.06% alginate and 0.9 mM calcium). 400 mL of the calcium alginate pregel was mixed with an 80 mL solution of poly-L-lysine (PLL, 187.5 mg, 7500 mw) in dH₂O. To the supernatant was added 240 mg of oligo ISIS 3521 (SEQ ID NO: 4) and stirred gently over 4 days. The resulting mixture proportions were 0.05% alginate, 0.75 mM Ca, 52.08 μm PLL (7500 mw) and 0.05% oligonucleotide. Microparticles formed in the mixture were measured after 4 days of stirring by laser scattering on a Horiba LA-910 analyzer to determine mean particle size of 127.991 μm (93.831 standard deviation).

After stirring, the 480 mL microparticle mixture was equally divided into twelve 45 mL (Falcon) tubes followed by centrifugaion for 30 minutes at 4000 rpm. The amount of oligonucleotide associated with or bound to PLL-alginate particles was determined from a 4×dilution sample by UV absorbance at γ=260 nm to be 176.41 mg (73.5%). To purify the complex from gree oligo, 32.5 mL of the clear supernatant was removed from each vial thus eliminating 51.67 mg of unbound oligonucleotide. The remaining 7.5 mL in each tube was then combined (90 mL total) and filtered through a 0.2μ membrane filter under vacuum eliminating a further 6.62 mg of unbound oligonucleotide. The remaining 40 mL solution comprising 176.41 mg (97.1%) bound oligo-microparticle complex and 5.3 mg (2.9%) unbound oligo was then lyophilized for storage prior to biological testing.

Bioavailability

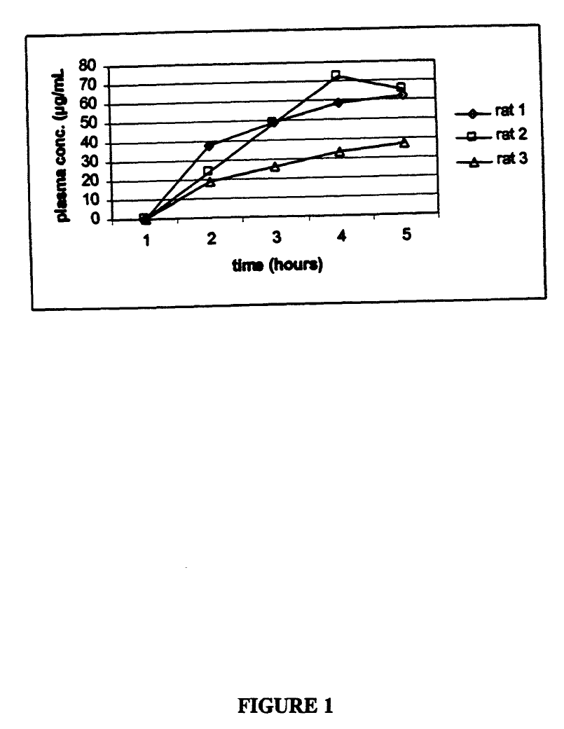

For in situ rat studies, 209.8 mg of the lyophilized oligo microparticulate and 225.0 mg of a lyophilized penetration enhancer mixture (PE) (sodium salts of CDCA, capric acid and lauric acid; 1:2:2) were combined with dH₂O and vortexed to remove air bubbles and create a homogeneous paste. 1 mL of the final formulation (10 mg oligo, 50 mg PE) was administered by intrajejeunal installation into three rats (kind, weight, age, sex). Blood samples of 300 mL were taken from femoral vein at time intervals of 30 minutes, 1, 2 and 3 hours post administration. 8 μL EDTA was added to the samples and plasma harvested after centrifugation. Plasma levels of oligo ISIS-3521 (SEQ ID NO: 4) were then determined by anion exchange HPLC, results shown in FIG. 1.

### EXAMPLE 2

PLGA particles with antisense oligonucleotide ISIS-2302 (SEQ ID NO: 2)

Formulation 2a

0.2 g of PLGA polymer was dissolved in 2 mL methylene chloride (CH₂Cl₂) and 0.1 of oligo ISIS-2302 (SEQ ID NO: 2) was dissolved in water (0.1 mL) along with 0.2g of DMRIE (a 1:1 mixture of lipid 1,2-dimyristyloxypropyl-3-dimethyl-hydroxy ethyl ammonium bromide and cholesterol). The aqueous solution was added to the polymer solution to give a dispersed phase. A continuous phase was prepared by dissolving 0.5 g of polyvinyl alcohol in 100 mL of water. The dispersed phase was then slowly added to the continuous phase while mixing and continued mixing for about 2 hours and increasing the temperature to about 40° C. to evaporate the volatile solvent.

Formulation 2b

0.1 g of PLGA polymer was dissolved in 1.0 mL HFA (hexafluoroacetone) and 9.9 mg of oligo ISIS-2302 (SEQ ID NO: 2) was dissolved in water. The aqueous and polymer solutions were combined to give a dispersed phase. A continuous phase was prepared by dissolving 2.1 g of sorbitan sesquioleate in 60 mL of cottonseed oil. The dispersed phase was then slowly added to the continuous phase while mixing and continued mixing for about 3 hours and increasing the temperature to about 50° C. to evaporate the volatile solvent.

Formulation 2c

0.2 g of PLGA polymer was dissolved in a mixture of 1.5 mL HFA and 0.5 mL ACN (acetonitrile) and 20 mg of oligo ISIS-2302 (SEQ ID NO. 2) was dissolved in water. The aqueous and polymer solutions were combined to give a dispersed phase. A continuous phase was prepared by dissolving 6 g of sorbitan sesquioleate in 200 mL of cottonseed oil. The dispersed phase was then slowly added to the continuous phase while mixing and continued mixing for about 1.5 hours at rt and increasing the temperature to 90° C. to evaporate the volatile solvent.

Formulation 2d

0.2 g of PLGA polymer was dissolved in 1.5 mL HFA and 22 mg of the calcium salt of oligo ISIS-2302 (SEQ ID NO: 2) was dissolved in water. The aqueous and polymer solutions were combined to give a dispersed phase. A continuous phase was prepared by dissolving 9 g of sorbitan sesquioleate in 150 mL of cottonseed oil. The dispersed phase was then slowly added to the continuous phase while mixing and continued mixing for about 2 hours and increasing the temperature to about 40° C. to evaporate the volatile solvent.

### EXAMPLE 3

Protamine Particles with Antisense Oligonucleotide ISIS-2302 (SEQ ID NO: 2)

Cationic protamine polymer was dissolved in water and mixed with an oligonucleotide solution comprising ISIS-2302 (SEQ ID NO: 2) and a complex modifier in water. The resulting precipitated particles were then separated by centrifugation or filtration. The specific modifier and relative amounts of the solution components are found in the table below.

TABLE I

oligo solution

| protamine solution | | | amount | water | oligo |
|---|---|---|---|---|---|
| protamine (mg) | water (mL) | modifier | (mg) | (mL) | (mg) |
| 10 | 1 | none | — | 0.5 | 5 |
| 90 | 1 | Na-alginate | 51 | 1 | 25 |
| 32^{a} | 1 | Na-alginate | 19 | 1 | 10 |
| 6 | 1 | trilysine | 6 | 1 | 5 |
| 2.5-5 | 0.5 | trilysine | 3-6 | 0.5 | 5 |
| 0.6 | 0.1 | CaCl₂ | 3.5 | 0.2^{b} | 1 |
| 53 | 1 | bovine albumin | 51 | 2 | 25 |
| 0.06 | 0.03 | glucosamine | 1-2 | 0.02 | 1 |
| 0.2 | 0.2 | lysine | 1 | 0.1 | 1 |
| 0.2 | 0.2 | dilysine | 1 | 0.1 | 1 |
| 0.2 | 0.2 | trilysine | 1 | 0.2 | 1 |
| 0.2 | 0.2 | arginine | 1 | 0.1 | 1 |
| 0.2 | 0.2 | histidine | 1 | 0.1 | 1 |
| 0.2 | 0.2 | glucosamine | 1 | 0.1 | 1 |
| 0.2 | 0.2 | galactosamine | 1 | 0.1 | 1 |
| 0.2 | 0.2 | Nicotinamide | 1 | 0.1 | 1 |
| 0.2 | 0.2 | Creatine | 1 | 0.1 | 1 |
| 0.4 | 0.02 | Arginine | 2 | 0.2 | 1 |
| 0.125-1.0 | 0.025-0.2 | None | — | 0.1 | 0.5 |
| 0.5 | 0.1 | lys ethyl ester | 5.5 | 0.1 | 5 |
| 0.01-1.0 | 0.1 | arg ethyl ester | 10 | 0.11 | 1 |

^{a}added CaCl2 to complete precipitation
^{b}CaC12 in 0.1 mL water added to oligo in 0.1 mL water

### EXAMPLE 4

Chitosan, Spermine and Arginine-Ethyl Ester Particles with Antisense Oligonucleotide ISIS-2302 (SEQ ID NO: 2)

Multiparticulate formulations comprising chitosan, spermine and arginine ethyl ester as carrier particles for ISIS-2302 (SEQ ID NO: 2) were prepared by mixing a cationic particle-forming material with ISIS-2302 (SEQ ID NO: 2) in water or saline. The specific components and amounts are as follows.

TABLE II

| particle material | oligo solution |
|---|---|
| chitosan (0.125-1.0 mg) in 0.05-0.4 mL H₂O | 0.5 mg in 0.1 mL H₂O |
| spermine (305 mg) in 2 mL PBS | 296 mg in 3.0 mL PBS |
| arg-ethyl ester (10-500 mg) in 1 mL | 5-50 mg in 1 mL H₂O |

What is claimed is:

1. A non-parenteral multi-particulate formulation comprising:
a plurality of carrier particles;
an oligonucleotide to be delivered across a mucosal membrane; and
a penetration enhancer selected from the group consisting of a fatty acid, bile salt, chelating agent and non-chelating non-surfactant,
wherein said fatty acid is selected from the group consisting of oleic acid, lauric acid, capric acid, myristic acid, palmitic acid, stearic acid, linoleic acid, linolenic acid, dicaprate, tricaprate, moolein, dilaurin, caprylic acid, arachidonic acid, glyceryl 1-monocaprate, 1-dodecylazacycloheptan-2-one, acylcarnitines, acylcholines, monoglycerides, diglycerides, and salts thereof.

2. The formulation of claim 1, wherein said oligonucleotide is an antisense oligonucleotide.

3. The formulation according to claim 2, wherein said oligonucleotide is selected from the group consisting of SEQ ID NO: 1, SEQ ID NO: 2, SEQ ID NO: 3, SEQ ID NO: 4, SEQ ID NO: 5, SEQ ID NO: 6 and SEQ ID NO: 7.

4. The formulation according to claim 1, wherein said carrier particles are bioadhesive.

5. The formulation according to claim 1, wherein said carrier particles comprise a particle-forming substance selected from the group consisting of poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches.

6. The formulation according to claim 1, wherein said carrier particles comprise a particle-forming material selected from the group consisting of chitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate),poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylaerylate, poly(D,L-lactic acid), poly(DL-lactic-co-glycolic acid (PLGA), and polyethyleneglycol (PEG).

7. The formulation according to claim 1, wherein said carrier particles are polycationic.

8. The formulation according to claim 1 wherein said carrier particles comprise a complex of poly-L-lysine and alginate; or protamine and alginate, lysine, dilysine, trilysine, calcium, albumin, glucosamine, arginine, galactosamine, nicotinamide, creatine, lysine-ethyl ester and arginine-ethyl ester.

9. The formulation according to claim 1, wherein said carrier particles are other than polycationic.

10. The formulation according to claim 9, wherein said carrier particles comprise poly(DL-lactic co-glycolic acid) (PLGA).

11. The formulation according to claim 1, wherein said carrier particles are miniparticles.

12. The formulation according to claim 1, wherein said carrier particles are microparticles.

13. The formulation according to claim 1, wherein said carrier particles are nanoparticles.

14. The formulation according to claim 1, wherein said penetration enhancer is selected from the group consisting of fatty acids, bile acids and salts and mixtures thereof.

15. The formulation according to claim 1, wherein said penetration enhancer is selected from UDCA, CDCA and salts and mixtures thereof.

16. The formulation according to claim 1, wherein said penetration enhancer is a mixture comprising the sodium salts of UDCA, capric acid and lauric acid.

17. The formulation according to claim 1 wherein said penetration enhancer is a component of said particle.

18. The formulation according to claim 1, wherein the surface of said carrier particle is substantially coated with said penetration enhancer.

19. The formulation according to claim 1, further comprising a mucolytic material.

20. The formulation according to claim 19, wherein said mucolytic material is selected from the group consisting of N-acetylcysteine, dithiothreitol, pepsin, pilocarpine, guaifenesin, glyceryl guaiacolate, terpin hydrate, ammonium chloride, guattenesin, ambroxol, bromhexine, carbocysteine, domiodol, letosteine, mecysteine, mesna, sobrerol, stepronin, tiopronin and tyloxapol.

21. The formulation according to claim 1 in a dosage form selected from the group consisting of tablets, capsules and filled gelcaps.

22. The formulation according to claim 21, further comprising an enteric material protecting the dosage form from degradation in a gastric environment.

23. The formulation according to claim 22, wherein said enteric material is selected from the group consisting of cellulose acetate phthalate (CAP), propylene glycol, EUDRAGIT and sorbitan monoleate.

24. The formulation according to claim 22, wherein said enteric material substantially coats the outer surface of the dosage form.

25. The formulation according to claim 22, wherein said enteric material substantially coats the outer surface of the individual carrier particles.

26. A method of delivering a biologically active substance across a mucosal membrane, comprising introducing to the mucosal membrane a multi-particulate formulation according to claim 1.

27. The method according to claim 26, wherein said biologically active substance is an oligonucleotide and said formulation is administered orally to a mammal.

28. A non-parenteral multi-particulate formulation comprising:
a plurality of carrier particles;
an oligonucleotide to be delivered across a mucosal membrane; and
a penetration enhancer selected from the group consisting of a fatty acid, bile salt, chelating agent and non-chelating non-surfactant.


SEQUENCE LISTING (9 sequences)

SEQ ID NO: 1 - DNA, 20 bp, Artificial Sequence
  Synthetic Oligonucleotide Sequence
tcccgcctgtgacatgcatt

SEQ ID NO: 2 - DNA, 20 bp, Artificial Sequence
  Synthetic Oligonucleotide Sequence
gcccaagctggcatccgtca

SEQ ID NO: 3 - DNA, 21 bp, Artificial Sequence
  Synthetic Oligonucleotide Sequence
gcgtttgctcttcttcttgcg

SEQ ID NO: 4 - DNA, 20 bp, Artificial Sequence
  Synthetic Oligonucleotide Sequence
gttctcgctggtgagtttca

SEQ ID NO: 5 - DNA, 20 bp, Artificial Sequence
  Synthetic Oligonucleotide Sequence
tccgtcatcgctcctcaggg

SEQ ID NO: 6 - DNA, 21 bp, Artificial Sequence
  Synthetic Oligonucleotide Sequence
gcgtttgctcttcttcttgcg

SEQ ID NO: 7 - DNA, 8 bp, Artificial Sequence
  Synthetic Oligonucleotide Sequence
ttggggtt

SEQ ID NO: 8 - DNA, 20 bp, Artificial Sequence
  Synthetic Oligonucleotide Sequence
gtgctcatggtgcacggtct

SEQ ID NO: 9 - DNA, 20 bp, Artificial Sequence
  Synthetic Oligonucleotide Sequence
gtgtgccagacaccctatct